(19) United States
(12) Patent Application Publication
(10) Pub. No.: US 2024/0058509 A1
(43) Pub. Date: Feb. 22, 2024

(54) BALLOON COMPOSITIONS FOR IMPLANT DEPLOYMENT

(71) Applicant: EDWARDS LIFESCIENCES CORPORATION, Irvine, CA (US)

(72) Inventors: Jeong Soo Lee, Diamond Bar, CA (US); Sean Chow, Irvine, CA (US); Tiffany Diemtrinh Tran, Anaheim, CA (US); Phu Vinh Le, Garden Grove, CA (US); Lynne Borowski Shwed, Park City, UT (US)

(21) Appl. No.: 18/499,995

(22) Filed: Nov. 1, 2023

Related U.S. Application Data
(63) Continuation of application No. PCT/US2022/026874, filed on Apr. 29, 2022.
(60) Provisional application No. 63/184,353, filed on May 5, 2021.

(51) Int. Cl. A61L 29/08; A61F 2/24; A61L 29/06; A61L 29/14

(52) U.S. Cl. CPC A61L 29/085; A61F 2/2433; A61L 29/06; A61L 29/143; A61M 2025/1075

(57) ABSTRACT

Balloon compositions that may be used for deployment of implants within a patient's body. The balloons in examples may be utilized for dilating implants and may be coupled to a delivery catheter for an implant. In examples, the balloons may be utilized to dilate other surfaces within the patient's body.

### CROSS-REFERENCE TO RELATED APPLICATIONS

This application is a continuation of International Application No. PCT/US2022/026874, filed Apr. 29, 2022, which designates the United States and was published in English by the International Bureau on Nov. 10, 2022 as WO2022/235495, which claims the benefit of U.S. Provisional Application No. 63/184,353, filed May 5, 2021, the entire contents of each of which are hereby incorporated by reference.

### BACKGROUND

A variety of maladies may affect an individual's body. Such maladies may be of the individual's heart, and may include maladies of the individual's heart valves, including the aortic, mitral, tricuspid, and pulmonary valves. Stenosis, for example, is a common and serious valve disease that may affect the operation of the heart valves and an individual's overall well-being.

Implants may be provided that may replace or repair portions of a patient's heart. Prosthetic implants, such as prosthetic heart valves, may be provided to replace a portion of a patient's heart. Prosthetic aortic, mitral, tricuspid, and even pulmonary valves may be provided.

Implants may be deployed to the desired portion of the patient's body percutaneously, in a minimally invasive manner. Such deployment may occur transcatheter, in which a catheter may be deployed through the vasculature of an individual.

During deployment of such implants, the implants must be dilated to provide an expanded configuration for such implant. Care must be taken to property dilate the implants to avoid over expansion or under expansion of such implants and to properly deploy such an implant. Care must also be taken to avoid rupture of a balloon utilized to deploy such an implant.

### SUMMARY

The present devices, systems, and methods may relate to balloon compositions that may be for deployment of implants within a patient's body. The balloons in examples may be utilized for dilating implants and may be coupled to a delivery catheter for the implant. In examples, the balloons may be utilized to dilate other surfaces within the patient's body.

The balloons may provide improved deployment of implants, including a reduced possibility of undesired movement of an implant during deployment. The balloons may have further benefits including an increased possibility of tear in a longitudinal dimension as opposed to a radial dimension, and reduced inflation pressure for the balloon. In examples, the balloons may have improved compliance properties to enhance the ease of a deployment procedure for the implant.

Examples herein may include a device for insertion within a portion of a patient's body. The device may comprise a balloon having a wall including an inner surface and a first layer and a second layer positioned adjacent to the first layer and not being thermally bondable to the first layer, wherein the balloon is expandable through pressure applied to the inner surface of the wall.

Examples herein may include a system for deploying an implant to a portion of a patient's body. The system may comprise a delivery catheter including an elongate shaft. The system may comprise a balloon coupled to the elongate shaft and configured for the implant to be positioned upon, and having a wall including an inner surface and a first layer and a second layer positioned adjacent to the first layer and not being thermally bondable to the first layer, wherein the balloon is expandable through pressure applied to the inner surface of the wall.

Examples herein may include a method of delivering an implant to a portion of a patient's body. The method may comprise inserting a balloon into the patient's body, the balloon having a wall including an inner surface and a first layer and a second layer positioned adjacent to the first layer and not being thermally bondable to the first layer. The method may comprise expanding the balloon through pressure applied to the inner surface of the wall to expand the implant at the portion of the patient's body.

Examples herein may include a crimping device for an implant for implantation within a portion of a patient's body. The crimping device may comprise a compressive body having an inner surface surrounding a channel for the implant to be positioned within, the inner surface having a tapered profile and configured to be contracted to apply a compressive force to the implant within the channel to crimp the implant. The crimping device may comprise an actuator for contracting the inner surface.

Examples herein may include a method for crimping an implant for implantation within a portion of a patient's body. The method may comprise inserting an implant within a channel of a compressive body, the compressive body having an inner surface with a tapered profile surrounding the channel. The method may comprise contracting the inner surface to apply a compressive force to the implant within the channel to crimp the implant.

Examples herein may include a device for insertion within a portion of a patient's body. The device may comprise a balloon having a wall including a blend of a semi-crystalline polymer and an amorphous polymer and having an inner surface, wherein the balloon is expandable through pressure applied to the inner surface of the wall.

Examples herein may include a system for deploying an implant to a portion of a patient's body. The system may comprise a delivery catheter including an elongate shaft. The system may comprise a balloon coupled to the elongate shaft and configured for the implant to be positioned upon, and having a wall including a blend of a semi-crystalline polymer and an amorphous polymer and having an inner surface, wherein the balloon is expandable through pressure applied to the inner surface of the wall.

Examples herein may include a method of delivering an implant to a portion of a patient's body. The method may comprise inserting a balloon into the patient's body, the balloon having a wall including a blend of a semi-crystalline polymer and an amorphous polymer and having an inner surface. The method may comprise expanding the balloon through pressure applied to the inner surface of the wall to expand the implant at the portion of the patient's body.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar examples.

FIG. 1 illustrates a perspective view of a tapered balloon according to an example of the present disclosure.

FIG. 2 illustrates a cross sectional view of the tapered balloon shown in FIG. 1.

FIG. 3 illustrates a side view of a delivery catheter according to an example of the present disclosure.

FIG. 4 illustrates a close-up view of a distal end of the delivery catheter shown in FIG. 3.

FIG. 5 illustrates a chart of a crimped outer diameter of an implant according to an example of the present disclosure.

FIG. 6 illustrates a rear perspective view of a crimper according to an example of the present disclosure.

FIG. 7 illustrates a front perspective view of the crimper shown in FIG. 6.

FIG. 8 illustrates a cross sectional schematic view of the interior surface of a crimping device according to an example of the present disclosure.

FIG. 9 illustrates an implant in the form of a valve crimped to a balloon according to an example of the present disclosure.

FIG. 10 illustrates the balloon shown in FIG. 9 partially expanded.

FIG. 11 illustrates a schematic view of an implant approaching an implantation site according to an example of the present disclosure.

FIG. 12 illustrates a schematic view of the implant shown in FIG. 11 deployed to an implantation site according to an example of the present disclosure.

FIG. 13 illustrates a cross sectional view of a balloon according to an example of the present disclosure.

FIG. 14 illustrates a chart of compliance of balloons.

FIG. 15 illustrates a chart of tear strength of balloons.

FIG. 16 illustrates a chart of maximum inflation pressure of balloons.

FIG. 17 illustrates a perspective view of an implant according to an example of the present disclosure.

FIG. 18 illustrates a top view of the implant shown in FIG. 17 according to an example of the present disclosure.

FIG. 19 illustrates a top view of the implant shown in FIG. 17 according to an example of the present disclosure.

FIG. 20 illustrates a cross sectional view of a tapered balloon.

### DETAILED DESCRIPTION

The following description illustrates some examples of the disclosure in detail. Those of skill in the art will recognize that there are numerous variations and modifications of the disclosure that are encompassed by its scope. Accordingly, the description of a certain example should not be deemed to limit the scope of the present disclosure.

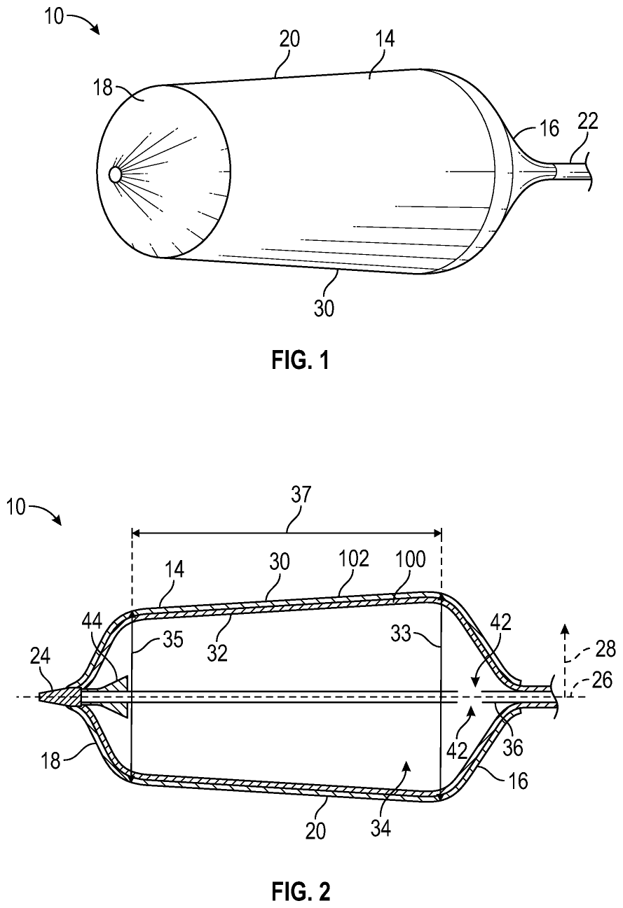

FIG. 1 illustrates a perspective view of a balloon 10 according to an example of the present disclosure. The balloon 10 may have a wall 14 and may be expandable through pressure applied to an inner surface of the wall 14. The balloon 10 may be configured for insertion within a portion of a patient's body.

The balloon 10, in examples, may include a first end portion 16 and a second end portion 18 and a length between the first end portion 16 and the second end portion 18. A central portion 20 may be positioned between the first end portion 16 and the second end portion 18.

The first end portion 16 may couple to a shaft 22, which may comprise an elongate shaft 22 of a delivery catheter according to examples herein. The first end portion 16 may taper outwardly in a distal direction towards the central portion 20.

The second end portion 18 may be positioned at a distal end of the central portion 20, and may taper inwardly in a distal direction to the end of the balloon 10. A nose cone 24 (marked in FIG. 2, yet not shown in FIG. 1) may be positioned at the end of the balloon 10 in examples, although a nose cone may not be utilized in certain examples.

The central portion 20 may be positioned between the first end portion 16 and the second end portion 18 and may be configured to apply a radially outward force to an implant that is positioned upon the central portion 20 or apply the radially outward force to another surface for dilation by the balloon 10.

The balloon 10 may have an elongate shape that extends along a longitudinal axis 26 (marked in FIG. 2) and may be symmetrical about the axis 26. The balloon 10 may be positioned radially outward from the longitudinal axis 26 and thus extends in a radial dimension 28 (marked in FIG. 2) outward from the longitudinal axis 26.

The balloon 10 may have a tapered profile. Specifically, as shown in FIGS. 1 and 2, the central portion 20 for applying the force to the implant or other surface, may have a tapered profile. The direction of the taper may vary according to the desired implementation of the balloon 10. For example, as shown in FIG. 2, the central portion 20 may taper inward in a distal direction. In examples however, the central portion 20 may taper outward in a distal direction. In examples, the central portion 20 and balloon 10 may lack a taper, and may have a cylindrical shape or another shape as desired. The taper may result in the central portion 20 having a larger diameter 33 at a proximal end than the diameter 35 at a distal end of the central portion 20 as shown in FIG. 2. The central portion 20 may have a length 37 along the longitudinal axis 26 as shown in FIG. 2.

FIG. 2 illustrates a cross sectional view of the balloon 10 shown in FIG. 1. The wall 14 of the balloon is shown in cross section to include an outer surface 30 facing opposite an inner surface 32. The outer surface 30 may comprise the portion of the balloon 10 that contacts an implant or a surface of another structure for dilation. In examples however, a coating or other structure may be positioned on the outer surface 30 of the wall 14 as desired, to form the outer surface of the balloon 10. The outer surface 30 at the central portion 20 may have a tapered profile.

The inner surface 32 may face an interior chamber 34 or fluid chamber that the wall 14 surrounds. The inner surface 32 may have pressure applied to it to expand the balloon 10. The interior chamber 34 may be configured to receive fluid for expanding the balloon 10. The interior chamber 34 for example may be filled with fluid, such as a liquid, to apply the pressure to the inner surface 32 and expand the balloon 10. The interior chamber 34 may be configured to receive fluid that fills the interior chamber 34 at a desired time, and may be configured for fluid to be withdrawn from the interior chamber 34 at a desired time to deflate the balloon 10.

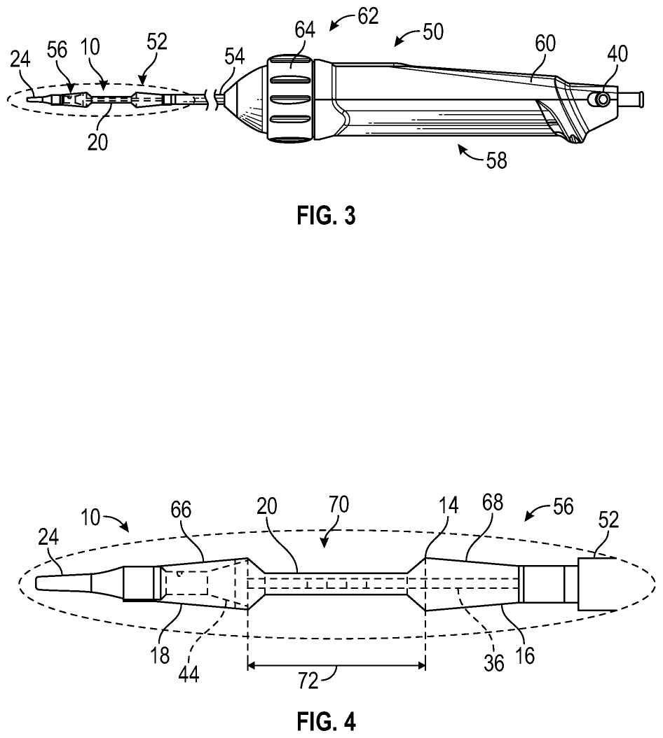

A fluid lumen 36, for example, may be configured to supply fluid to the interior chamber 34 and withdraw fluid from the interior chamber 34. The fluid lumen 36 may extend centrally within the balloon 10 along the longitudinal axis 26 as shown in FIG. 2, or may have another configuration as desired. A proximal end of the fluid lumen 36 may extend to a fluid port 40 of a delivery catheter as shown in FIG. 3 for example, or to another location as desired.

The fluid lumen 36 may include one or more channels 42 that may be utilized to supply fluid to or from the interior chamber 34 from the fluid lumen 36. In examples, the channels 42 may be positioned proximally with respect to the interior chamber 34, although other locations may be utilized in examples as desired.

The fluid lumen 36 may comprise a central shaft that extends through the balloon 10, or may end at a proximal portion of the balloon 10. One or more shafts may extend through the balloon 10 to the second end portion 18 or distal end portion of the balloon 10. The shafts, for example, may include the fluid lumen 36 or may include other structures such as a guide wire lumen (not shown). The shafts may extend to and couple with the nose cone 24 or other structure positioned at the distal end portion of the balloon 10. In examples, a structure such as a distal shoulder 44 may be coupled to a shaft.

FIGS. 1 and 2 illustrate the balloon 10 in an inflated or expanded state, in which the balloon 10 is filled with fluid. A tapered profile of the balloon 10 is visible.

FIG. 3 illustrates a side view of an example of the balloon 10 in a deflated or unexpanded state, along with a delivery catheter 50 that may include the balloon 10. The delivery catheter 50 as shown in FIG. 3 may include an elongate shaft 52 having a proximal end portion 54 and a distal end portion 56. The elongate shaft 52 may comprise an elongate body that may be flexible to allow for deflection of the elongate shaft 52 upon insertion into a patient's body.

The proximal end portion 54 of the elongate shaft 52 may couple to a housing in the form of a handle 58 that may be configured to be gripped by a user to control operation of the elongate shaft 52. The handle 58 may be manipulated to cause the elongate shaft 52 to be advanced or retracted within a patient's body to place the elongate shaft 52 in the desired orientation relative to an implantation site. The handle 58 may include an outer surface 60 configured to be gripped.

A control mechanism 62 may be included with the handle 58 and may be configured to be operated to deflect the elongate shaft 52 in examples. The control mechanism 62, for example, may comprise one or more actuators in the form of control knobs 64 or other actuators that may be utilized to deflect the elongate shaft 52. The control mechanism 62 may include pull tethers or other structures that may be utilized to deflect the elongate shaft 52 via tension applied to the pull tethers. In examples, other forms of control mechanisms may be utilized as desired.

A fluid port 40 may be positioned on the handle 58 that may be configured to pass fluid to or withdraw fluid from the fluid lumen 36 shown in FIG. 2 for example. The fluid port 40 may couple to a fluid actuator or other device utilized to fill or withdraw fluid from the interior chamber 34 shown in FIG. 2.

The distal portion or distal end portion 56 of the elongate shaft 52 may include the balloon 10. The balloon 10 is shown in a deflated or unexpanded state, which may be a state for insertion of the elongate shaft 52 into the patient's body with an implant positioned thereon.

FIG. 4, for example, illustrates a close up view of the distal end portion 56, showing the balloon 10 in the deflated or unexpanded state. Positions of the first end portion 16, the second end portion 18, and the central portion 20 are shown relative to the position of a central shaft, which may comprise the fluid lumen 36. The second end portion 18 is shown to comprise a distal shoulder 66 positioned over the distal shoulder 44 of the central shaft, and the first end portion 16 is shown to comprise a proximal shoulder 68. An implant retention area 70 may include the central portion 20 of the balloon 10 and may be positioned between the distal shoulder 66 and the proximal shoulder 68. The implant retention area 70 may have a length 72, and may have a diameter that is less than a diameter of the distal shoulder 66 and the proximal shoulder 68.

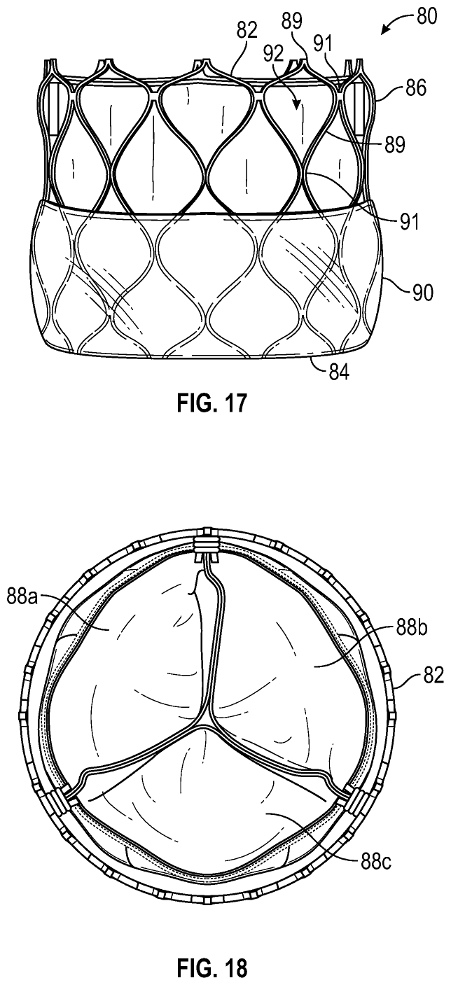

The implant that may be positioned at the implant retention area 70 may have a variety of forms. FIGS. 17-19 for example, illustrate a form of an implant 80 that may be utilized according to examples herein. FIG. 17 illustrates a perspective view of the implant 80, in the form of a prosthetic heart valve. The prosthetic implant 80 may be configured to be deployed within a portion of a patient's body. The prosthetic implant 80, for example, may be deployed within a native heart valve annulus, which may comprise a native aortic valve, or in examples may comprise a native mitral, tricuspid, or pulmonary valve. In examples, the implant 80 may have other forms, and may comprise a stent or other form of medical implant as desired.

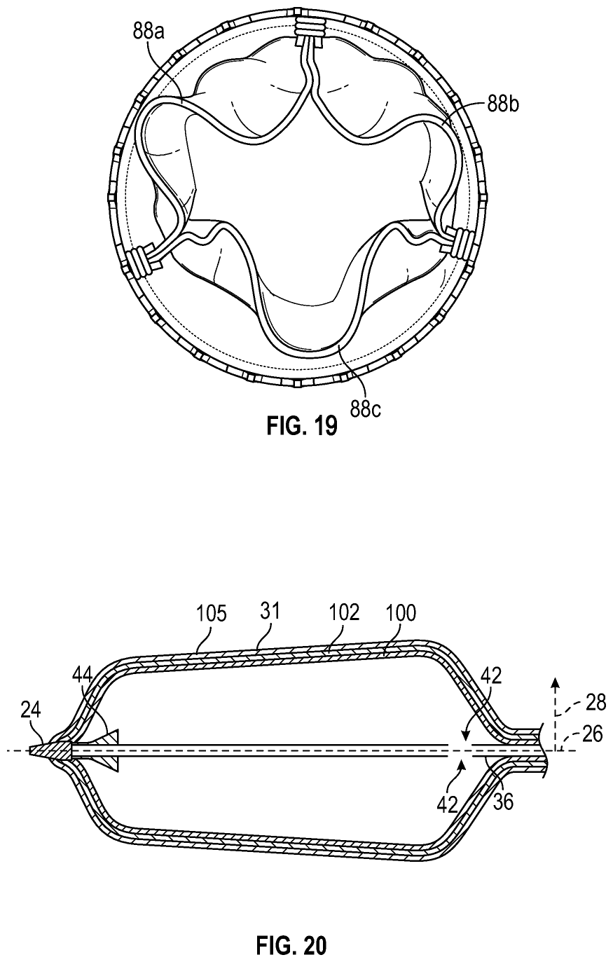

The prosthetic implant 80 may include a proximal or first end 82 and a distal or second end 84, and a length therebetween. The prosthetic implant 80 may include a body in the form of a frame 86. The prosthetic implant 80 may further include one or more of a plurality of leaflets 88a-c (marked in FIGS. 18 and 19) coupled to the frame 86 and may include a skirt 90 covering an outer surface of a distal portion of the frame 86. The leaflets 88a-c may move back and forth between open and closed positions or states or configurations to replicate the motion of a native valve. The leaflets 88a-c may extend inward from an inner surface of the implant 80 that the balloon 10 may exert a force against to dilate the implant 80.

The leaflets 88a-c may be configured to open and close during operation such that the first end 82 of the implant 80 forms an outflow end of the implant 80, and the second end 84 of the implant 80 forms an inflow end of the implant 80. The leaflets 88a-c may be configured to impede fluid flow in an opposite direction from the outflow end to the inflow end of the implant 80 when the leaflets 88a-c are in a closed position.

The frame 86 may comprise a plurality of struts 89 connected at junctures 91. A plurality of openings 92 may be positioned between the struts 89. The openings 92 may be configured to reduce the overall weight of the frame 86, and also allow the frame 86 to be compressed to reduce a diameter of the frame 86 and be expanded to increase a diameter of the frame 86. The frame 86 may be configured to be radially compressed and axially lengthened while being radially compressed. The struts 89 may be configured such that as the frame 86 is compressed to reduce a diameter of the frame 86, the length of the frame 86 may increase. Also, as the frame 86 is expanded to increase the diameter of the frame 86, the length of the frame 86 may decrease. The frame 86 may be compressed in a variety of manners, including use of a crimping device, and may be expanded in a variety of manners, including being expanded with a balloon as disclosed herein.

The configuration of the implant shown in FIGS. 17-19 may be varied in examples.

Referring back to FIG. 4, the use of a balloon 10 having a tapered shape may provide benefits in the crimped profile of an implant that is positioned at the implant retention area 70 and may include other benefits such as improved flow of inflation fluid to inflate the balloon 10 and dilate an implant positioned upon the implant retention area 70 of the balloon 10.

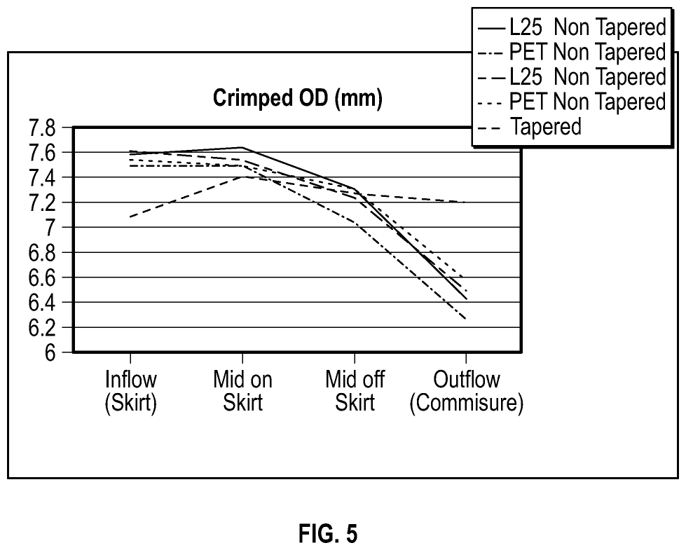

For example, FIG. 5 illustrates a chart of a crimped outer diameter of an implant, such as the implant shown in FIGS. 17-19 upon a variety of different types of balloons. The Y-axis shows outer diameter in millimeters and the X-axis shows a position along the implant. Upon an implant being crimped to a non-tapered balloon, whether the balloon is made of a material such as Grilamid L25 or polyethylene terephthalate (PET), the inflow side or distal end of the implant including a skirt has a larger diameter than the outflow side or proximal end of the implant. This is because the skirt, such as the skirt 90 shown in FIG. 17, has increased bulk relative to the frame 86 alone, and increases the diameter at the inflow or distal side of the implant. A tapered balloon as shown in FIGS. 1 and 2, for example, may have increased material and diameter at the outflow or proximal side of the implant even in a deflated state, thus resulting in an implant with a linear outer diameter along the length of the implant, as shown in the dashed lines in FIG. 5, upon being crimped to the balloon.

A benefit to a tapered shape may further include improved flow of inflation fluid to inflate the balloon 10 and dilate an implant positioned upon the implant retention area 70 of the balloon 10. The balloon 10, having a tapered profile as shown in FIG. 2, may allow for enhanced flow of fluid from the proximal portion or first end portion 16 in a direction towards the second end portion 18, or distal portion as shown in FIG. 2 during inflation. The enhanced flow of fluid may be caused by a larger diameter of the interior chamber 34 at the proximal or first end portion 16 of the balloon 10 shown in FIG. 2, thus allowing for enhanced fluid flow to the second end portion 18 or distal end portion shown in FIG. 2.

The enhanced flow of fluid may allow for more symmetric inflation of the balloon 10 and deployment of the implant at both the first end 82 and the second end 84 of the implant. FIG. 10, for example, illustrates the balloon 10 being inflated in which the first end portion 16 of the balloon 10 and the second end portion 18 of the balloon 10 both inflate at a similar rate, to form a dumbbell shape for the balloon 10. The implant 80 positioned between the ends of the dumbbell deploys with a bowed shape, at both ends 82, 84 of the implant 80. As such, the implant 80 has a reduced possibility of slipping longitudinally along the outer surface of the balloon 10 and possibly being misdeployed during inflation of the balloon 10, due to the larger size of the ends 16, 18 of the balloon 10 and a symmetrical inflation of the ends 16, 18. The ends 16, 18 may inflate at a similar rate at a similar time, to impede a longitudinal sliding movement of the implant 80. The ends 16, 18 may inflate at a similar rate at a similar time due to the enhanced flow of fluid from the proximal portion or first end portion 16 in a direction towards the second end portion 18, or distal portion as shown in FIG. 2 during inflation. Use of a tapered balloon may produce other benefits in examples.

The balloon 10 may have a composition that may provide a variety of benefits, including enhancing the ability of the balloon 10 to retain a tapered profile. For example, referring to FIG. 2, the wall 14 may include a first layer 100 and a second layer 102 that is positioned adjacent to the first layer 100 and is not thermally bondable to the first layer 100. The second layer 102 may be incompatible or immiscible with the first layer 100. The first layer 100 may be positioned radially inward of the second layer 102 in examples, and may comprise the inner surface 32 of the wall 14 in examples. The second layer 102 may be positioned radially outward of the first layer 100 and in examples may comprise the outer surface 30 of the wall 14 in examples.

The first layer 100 may be configured to be thermally bonded to the elongate shaft 52 of the delivery catheter 50 in examples. The first layer 100, for example, may comprise a polyamide or a co-polyamide or another material as desired. The first layer 100, for example, may comprise nylon, such as nylon 12, or a material such as Pebax® or Grilamid L25. The first layer 100 may comprise aliphatic polyamide, aromatic polyamide, polyamide 12, polyamide 11, or co-polyamide, or other materials. The elongate shaft 52 of the delivery catheter 50 may be made of a same or similar material for the first layer 100, such that the elongate shaft 52 is thermally bonded to the first layer 100. The thermal bonding may couple the first layer 100 to the elongate shaft 52. Other materials may be utilized for the first layer 100 as desired.

The second layer 102 may be configured to be thermally non-bondable with the first layer 100 and the elongate shaft 52 of the delivery catheter in examples. The second layer 102, for example, may comprise a polyester or another material as desired. The second layer 102, for example, may comprise a polyethylene terephthalate (PET), a polybutylene terephthalate (PBT) or a thermoplastic elastomer copolyester (such as Hytrel®), or combinations thereof in examples. Other materials may be utilized for the second layer 102 as desired.

The first layer 100 and second layer 102 being thermally non-bondable may allow the layers to separate from each other if the wall 14 were cut and the layers 100, 102 were pulled from each other. The first layer 100 may extend over the second layer 102 during the formation process of the wall 14, yet may remain thermally non-bondable to the second layer 102 and unbonded to the elongate shaft 52 of the delivery catheter.

The presence of the second layer 102 may provide additional burst pressure and tear resistance. Further, puncture resistance may be provided due to the presence of the second layer 102.

The overall thickness of the second layer 102 may comprise 25 percent to 50 percent of the total wall 14 thickness in examples, although other configurations may be utilized as desired.

A compliance of the balloon 10 is an increase in diameter of the balloon 10 with pressure. The second layer 102 may comprise a material that has less compliance than the first layer 100. The first layer 100, for example, may comprise a semi-compliant material and the second layer 102 may comprise a low compliance material, although other configurations may be utilized as desired.

A balloon 10 as disclosed herein may have an outer diameter compliance between 0.30 millimeters per atmosphere (mm/atm) and 0.42 millimeters per atmosphere (mm/atm). The compliance may be in a range between four atmospheres to eight atmospheres applied to the balloon 10 in examples. A balloon 10 as disclosed herein may have a normalized compliance (which is compliance in millimeters per atmosphere per balloon outer diameter in millimeters) of between 10 percent to 25 percent. In examples, the wall 14 may have an outer diameter compliance of between 10 percent growth per atmosphere and 16 percent growth per atmosphere between 4 atmospheres to 8 atmospheres when normalized by a diameter of the balloon. Other values of compliance may be utilized in examples as desired.

In examples, a burst pressure of the balloon 10 may be at least about 7.5 atmospheres. In examples, the wall 14 may have a hoop strength (which is burst pressure times diameter divided by twice the thickness) of at least 22,000 pounds per square inch or at least 24,000 pounds per square inch in examples. Other configurations of the balloon 10 may be utilized as desired.

The taper of the central portion 20 in examples may be a difference in the diameter 35 at the distal end of the central portion 20 (marked in FIG. 2) to the diameter 33 at the proximal end of the central portion 20 (marked in FIG. 2) of at least 1 millimeter, or in examples, at most 3 millimeters at a nominal inflation pressure of 4 atmospheres. The taper may be in a range between 1 millimeter and 3 millimeters in examples at a nominal inflation pressure of 4 atmospheres. Other dimensions may be utilized in examples as desired.

The use of a relatively low compliance material (for example, the material of the second layer 102) may enhance the strength of the balloon 10, thus allowing the balloon 10 to retain a tapered shape as disclosed herein. The structure and features of the balloon 10 disclosed herein, however, may be utilized with another shape of balloon, such as a cylindrical balloon or other shape in examples as desired.

The wall 14 of the balloon 10 in examples may be formed by the layers 100, 102 being co-extruded and blow molded. The blow molding may include pressuring the wall 14 while stretching the wall 14 axially. Such a formation process may provide benefits resulting from the configuration of the second layer 102. For example, such a process may align the molecules of the second layer 102 material to enhance strength in the radial dimension (indicated in FIG. 2 with reference number 28) relative to an axial dimension along the longitudinal axis 26.

The tear strength for the wall 14 in the axial dimension accordingly may be weaker than in the radial dimension. For example, the tear strength of the wall 14 in the radial dimension may be at least 20 percent higher than the tear strength of the wall 14 in the axial dimension, although other configurations may be utilized as desired. Such a feature may be desirable in the event of an accidental burst of the balloon 10. If the tear strength of the wall 14 in an axial direction is weaker than the tear strength of the wall 14 in a radial direction then the wall 14 is more likely to tear longitudinally. A longitudinal tear may be desirable over a radial tear because a radial tear may impede the ability of the burst balloon to be retracted into a catheter sheath for removal from the patient's body. For example, a radial tear may form an umbrella or tented shape with a large diameter that may not fit into a smaller diameter catheter sheath for retrieval. As such, a longitudinal tear in the case of an accidental burst may be desirable for retrieval of an accidentally burst balloon.

Various other configurations of the wall 14 may be provided. For example, in one example the second layer 102 may comprise an intermediate layer between the first layer 100 and a third layer that may be positioned radially outward of the second layer 102. FIG. 20, for example, illustrates a third layer 105 that may be positioned upon the second layer 102. The third layer 105 may comprise the outer surface 31 of the balloon 10 and may be made of the same or similar materials as the first layer 100. The third layer 105, for example, may be made of a polyamide or a co-polyamide or another material as desired. The third layer 105, for example, may comprise nylon, such as nylon 12, or a material such as Pebax® or Grilamid L25. The third layer 105 may be made of a material that is thermally bondable with the first layer 100, yet is not bonded to the first layer 100 due to the presence of the second intermediate layer 102. The presence of the third layer 105, for example, may provide a higher burst pressure of at least about 9.15 atmospheres. Other configurations may be utilized as desired.

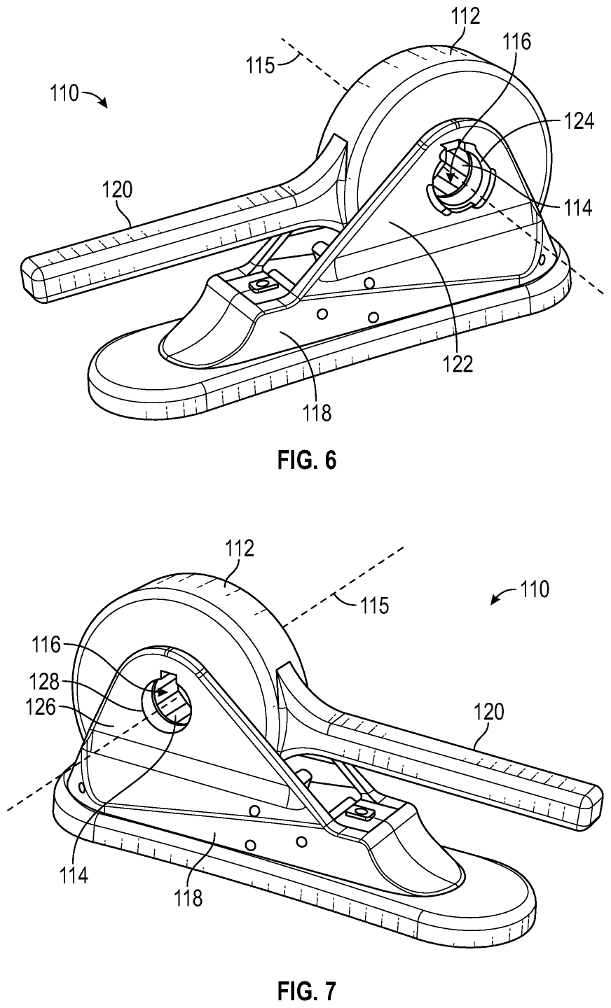

A crimping device 110 may be utilized to crimp an implant to the balloon 10. The crimping device 110 may be for an implant for implantation within a portion of the patient's body. The implant, for example, may be configured similarly as the implant 80 shown in FIGS. 17-19, although other configurations may be utilized as desired. FIG. 6 illustrates a rear perspective view of a crimping device 110 that may be utilized according to examples herein. The crimping device 110 may be utilized to crimp an implant to a balloon that has a tapered profile, similar to the balloon 10 shown in FIG. 2 for example.

The crimping device 110 may include a compressive body 112 having an inner surface 114 that surrounds a channel 116 for the implant to be positioned within. The inner surface 114 may extend around a longitudinal axis 115 that the channel 116 extends along. The compressive body 112 may be positioned upon a base 118 and may be coupled to an actuator 120 for contracting the inner surface 114. The actuator 120, for example, may comprise a lever or another form of actuator for contracting the inner surface 114.

The crimping device 110 may include a rear face 122 including a rear opening 124 for the implant to be passed through to be positioned within the channel 116.

FIG. 7 illustrates a front perspective view of the crimping device 110. The crimping device 110 may include a front face 126 facing opposite the rear face 122, and including a front opening 128 for the implant to be passed through to enter the channel 116.

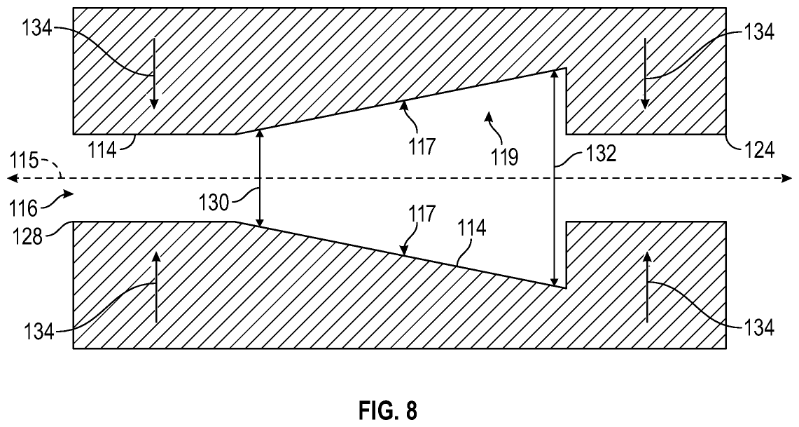

FIG. 8 illustrates a cross sectional view of the inner surface 114 of the crimping device 110 taken along the longitudinal axis 115. The inner surface 114 is shown to have a tapered profile. The tapered profile may include one or more linear surfaces 117 as shown in FIG. 8, or may include surfaces that are curved if desired. A central portion 119, between the end portions of the inner surface 114, is shown to include the tapered profile.

The smaller diameter 130 of the tapered profile in examples may be positioned at the smaller diameter 35 of a tapered balloon as shown in FIG. 2 for example. The larger diameter 132 of the tapered profile may be positioned at the larger diameter 33 of the tapered balloon as shown in FIG. 2 for example. The smaller diameter 130 in examples may be positioned at the portion of the implant 80 including the skirt 90. The skirt 90, for example, may comprise a larger diameter portion of the implant and the smaller diameter 130 portion may compress this portion of the implant to a greater amount. The channel 116 may be configured to receive the balloon 10 therein, and the inner surface 114 is configured to crimp the implant 80 to the balloon 10.

The inner surface 114 having the tapered profile is configured to be contracted to apply a compressive force to the implant within the channel 116 to crimp the implant. The implant 80 upon the balloon 10 may be inserted within the channel 116 of the compressive body 112. The inner surface 114 may be contracted to apply a compressive force to the implant within the channel to crimp the implant. The implant may be crimped to the balloon 10. The compressive force of the inner surfaces 114 shown in FIG. 8 may be radially inward towards the longitudinal axis 115 (as indicated with arrows 134 in FIG. 8). The actuator 120 shown in FIG. 7 for example may be gripped and rotated to cause the inner surfaces 114 to be pressed towards each other to compress the implant. The actuator 120 may be retracted to cause the inner surfaces 114 to move away from each other to allow the crimped implant to be removed from the channel 116.

In examples, the direction of the taper of the tapered profile may be reversed from the direction shown in FIG. 8. The reversed direction may accommodate an implant placed with the skirt 90 positioned at the larger diameter 33 of the tapered balloon as shown in FIG. 2 for example.

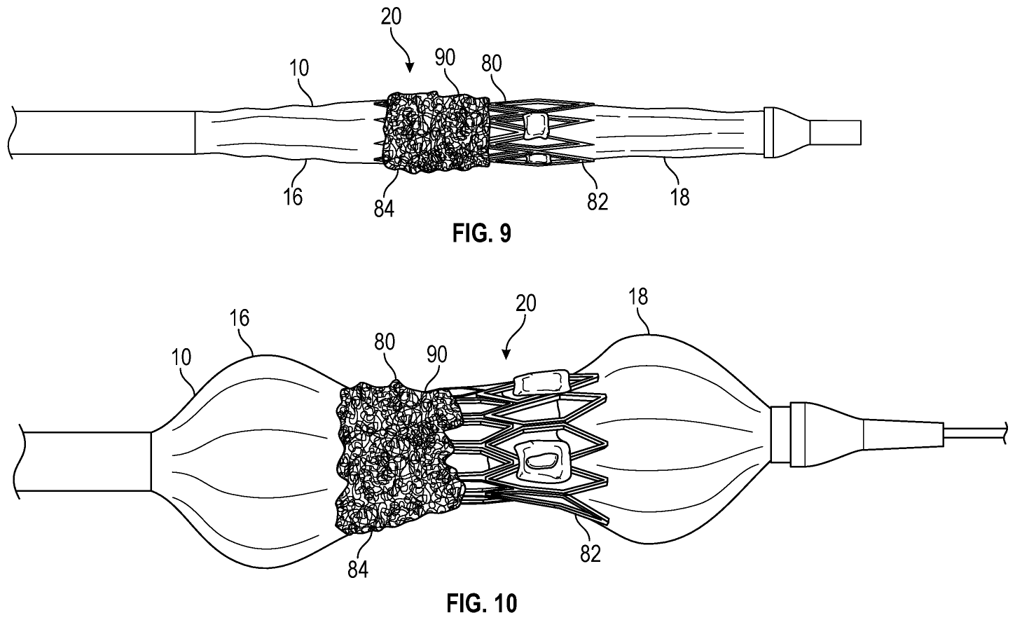

FIG. 9 for example, illustrates the implant 80 crimped to the balloon 10 with the skirt 90 positioned at the larger diameter 33 of the tapered balloon 10. The taper of the balloon may yet allow the implant 80 to be deployed with a bowed shape as shown in FIG. 10 for example. FIG. 10 illustrates the balloon 10 partially inflated. The presence of the first end portion 16 and the second end portion 18 of the balloon 10 may allow the implant 80 to remain positioned between the end portions 16, 18 and not slip off of the balloon 10 longitudinally or otherwise be undesirably displaced during deployment of the implant 80. The larger end portions 16, 18 positioned adjacent a narrower central portion 20 may impede the longitudinal movement of the implant 80, to allow for a more precise and predictable deployment position of the implant 80. The balloon 10, for example, may have a dumbbell or hourglass shape during deployment, as shown in FIG. 10.

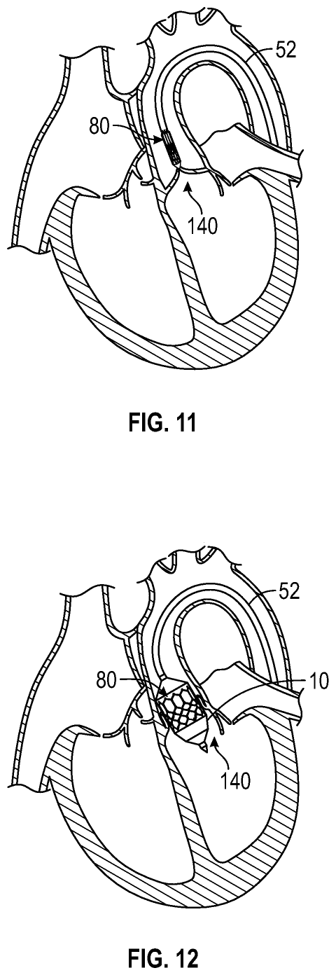

FIGS. 11 and 12 illustrate an exemplary operation of deploying the implant 80 in the form of a prosthetic heart valve. The balloon 10 and the elongate shaft 52 of the delivery catheter 50 may be inserted into a patient's body. The insertion may be transvascular in examples, and may be via a femoral entry, or other forms of entry in examples. The balloon 10 and elongate shaft 52 may travel over the aortic arch in examples, although other approaches (e.g., transapical, transseptal, among others) may be utilized.

The implant 80 for example, may be positioned at the inflation site, which may be an aortic valve 140 as shown herein, or another location as desired. The balloon 10 may be expanded through pressure applied to the inner surface of the wall of the balloon to expand the implant at the portion of the patient's body comprising the inflation site or implantation site.

FIG. 12 illustrates the balloon 10 being inflated. The implant 80 is expanded upon the balloon 10 and deployed to the implantation site. The balloon 10 may then be deflated and withdrawn from the patient's body with the implant 80 remaining in position. The implant 80 may be delivered to the implantation site upon the balloon 10 in examples, or may be advanced to the implantation site and then slid onto the balloon 10 for deployment in examples.

The implantation may be to a native valve or may be to another prior deployed prosthetic valve. For example, the balloon 10 may be utilized in a valve-in-valve procedure in which the implant 80 is deployed within a previously deployed prosthetic valve.

The balloon 10 may be configured to dilate an expandable implant positioned upon the wall of the balloon in examples, or may be configured to dilate another surface within the patient's body. For example, the balloon 10 may be utilized for dilation of surfaces of a structure such as native heart valve leaflets prior to implantation of the implant 80, or may be utilized for dilation of surfaces of vessels or other surfaces within the patient's body.

Various configurations of the balloon 10 may be utilized as desired. The composition of the balloon 10 disclosed herein may be utilized with a tapered balloon, or another form of balloon such as a cylindrical balloon as desired. Components of systems disclosed herein may be utilized separately as desired.

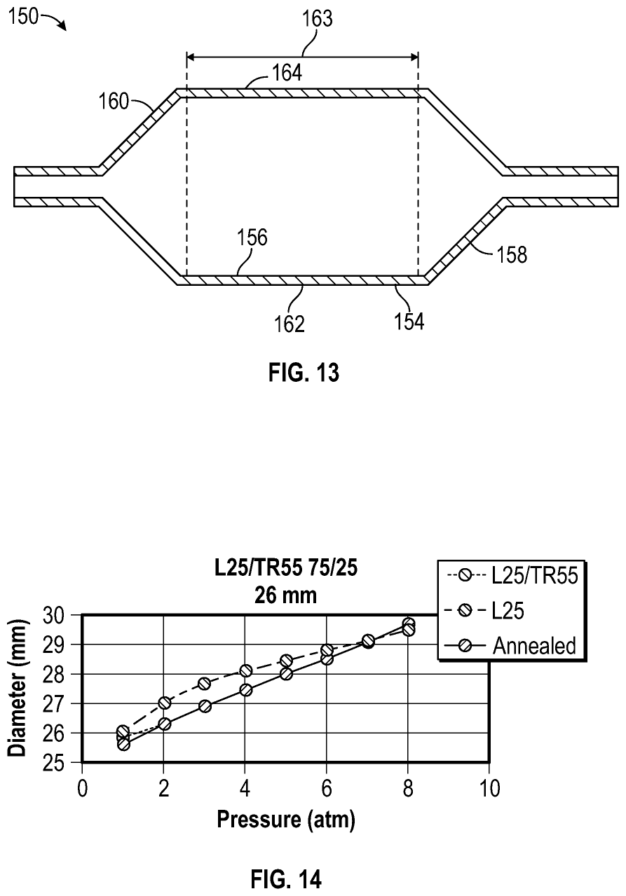

FIG. 13 illustrates an example of a balloon 150 that may be utilized according to examples herein. The balloon 150 may include a wall 154 including a blend of a semi-crystalline polymer and an amorphous polymer. The wall 154 may have an inner surface 156 and the balloon 150 may be expandable through pressure applied to the inner surface 156 of the wall 154.

The balloon 150 may be configured similarly as the balloon 10 shown in FIG. 2, and may include a first end portion 158, a second end portion 160 and a central portion 162 positioned between the first end portion 158 and the second end portion 160. The end portions 158, 160 may be tapered in a similar manner as the end portions 16, 18, or may have another configuration as desired. The central portion 162 may have a cylindrical shape as shown in FIG. 13, or may have a tapered shape or other shape as desired. The balloon 150, for example, may have a tapered profile as disclosed herein. The central portion 162 may have a length 163 between the ends of the central portion 162.

The wall 154 may have an outer surface 164 that is configured to apply a force to an implant or another surface to dilate such surface, as described with regard to the balloon 10. The balloon 150 may be configured for an implant to be positioned upon.

The balloon 150 in examples may be coupled to an elongate shaft of a delivery catheter in a similar manner as the balloon 10. In examples, the balloon 150 may be coupled to another form of device.

The blend of the semi-crystalline polymer and an amorphous polymer with the balloon 150 may produce desirable results over a balloon 150 including merely a semi-crystalline polymer. In examples, the semi-crystalline polymer may comprise a semi-crystalline polyamide. The semi-crystalline polymer may comprise semi-crystalline nylon, and may comprise semi-crystalline nylon 12, and may comprise a high viscosity polyamide 12. Other forms of material may be utilized as desired.

In examples, the semi-crystalline polymer may comprise Grilamid L25. Grilamid L25 may comprise a high viscosity, tasteless polyamide 12 (PA 12) grade. It may be formed by the polymerisation of laurolactam (lactam 12, obtained from the basic raw material butadiene), a ring-shaped, long-chain monomer with 12 carbon atoms. It may exhibit lower water absorption than PA 6, excellent dimensional stability, high toughness even at low temperatures, high resistance to chemicals, hydrolysis and weathering. It may possess a low density or lowest density among all polyamides, and may have good impact strength at low temperatures and easy processability. Other forms of materials having similar properties may be utilized as desired.

In examples, the amorphous polymer may comprise an amorphous polyamide. The amorphous polymer may comprise an amorphous nylon in examples. The amorphous polymer may comprise a transparent amorphous polyamide in examples and may comprise a transparent amorphous nylon in examples. In examples the amorphous polymer may comprise a transparent amorphous nylon with aromatic or cycloaliphatic units in examples.

In examples, the amorphous polymer may comprise Grilamid TR55. Grilamid TR55 may comprise a high purity transparent polyamide grade based on aliphatic and cycloaliphatic monomers. It may be obtainable from bis (3-methyl-4-aminocyclohexyl) methane, isophthalic acid and [omega] aminododecanoic acid or its lactam, among other materials. It may possess very high flexural fatigue strength, high colorability, good stiffness, and good toughness even at low temperatures and good dimensional stability. It may provide clear transparency even in high wall thicknesses, good resistance to chemicals and stress cracking. It may offer low moisture absorption, low water absorption compared to standard polyamides, high heat distortion temperature and easy processability. Other forms of materials having similar properties may be utilized as desired.

The wall 154 including the blend may be formed by blow molding and biaxial stretching (pressure and axial stretching). The tapered portions of the wall 154 may be formed by taper stretching.

The semi-crystalline polymer may have a melting temperature that is higher than a glass transition temperature of the amorphous polymer. For example, the melting temperature of the semi-crystalline polymer may be about 178 degrees Celsius, and the melting temperature of the amorphous polymer may have a glass transition temperature of about 160 degrees Celsius. It is believed that such characteristics may allow the polymer chains of the amorphous polymer to orient during a blow molding and stretching process easier than with a standard semi-crystalline polymer, and thus may result in improved radial tear resistance as opposed to longitudinal tear resistance.

In examples, the semi-crystalline polymer may have a melting temperature that is higher than the glass transition temperature of the amorphous polymer by at least 10 degrees Celsius, or another amount as desired.

The tensile modulus of the amorphous polymer may be greater than the tensile modulus of the semi-crystalline polymer. For example, the semi-crystalline polymer may have a tensile modulus of about 1100 MPa at room temperature, and the amorphous polymer may have a tensile modulus of about 2200 MPa at room temperature. Different amounts may be utilized for different materials utilized.

A blend of the semi-crystalline polymer and the amorphous polymer may be at different weights relative to each other. In examples, the semi-crystalline polymer may be at least 50 percent by weight of the total weight of the blend. In examples, the semi-crystalline polymer may be in a range of 65 percent to 90 percent of the total weight of the blend, and the amorphous polymer may be in a range of 10 percent to 40 percent of the total weight of the blend. Various other proportions may be utilized as desired.

The semi-crystalline polymer, for example, may comprise about 75 percent by weight of the total weight of the blend, and the amorphous polymer may comprise about 25 percent by weight of the total weight of the blend in examples. The semi-crystalline polymer, for example, may comprise about 50 percent by weight of the total weight of the blend, and the amorphous polymer may comprise about 50 percent by weight of the total weight of the blend in examples. The semi-crystalline polymer, for example, may comprise about 90 percent by weight of the total weight of the blend, and the amorphous polymer may comprise about 10 percent by weight of the total weight of the blend in examples.

The blend of the semi-crystalline polymer and the amorphous polymer may produce improved compliance of the balloon 150 compared to a balloon that only includes a semi-crystalline polymer.

FIG. 14, for example, illustrates a compliance chart of a blend of semi-crystalline polymer that is about 75 percent by weight of the total weight of the blend, and the amorphous polymer that is about 25 percent by weight of the total weight of the blend. A diameter of the balloon 150 is shown on the Y-axis and pressure is shown on the X-axis. The balloon 150 comprises a 26 millimeter diameter balloon. The semi-crystalline polymer is Grilamid L25 and the amorphous polymer is Grilamid TR55.

The compliance of the balloon 150 is about 0.5 millimeters per atmosphere, particularly in the range of 4 atmospheres to 8 atmospheres, which may be a working range of the balloon 150 during inflation. The compliance is improved relative to a balloon including only the semi-crystalline polymer (Grilamid L25) as shown in long dashed lines. The compliance is further shown to be linear. Annealing the blend of materials produces a similar compliance curve than a non-annealed version.

The compliance in examples may be an outer diameter compliance of the wall 154 of between 0.43 millimeters per atmosphere and 0.56 millimeters per atmosphere. The wall 154 may have an outer diameter compliance of between 17 percent growth per atmosphere and 21 percent growth per atmosphere between 4 atmospheres to 8 atmospheres when normalized by a diameter of the balloon. Various other amounts of compliance may be utilized in examples.

The outer surface of the balloon 150 having the blend may be less tacky and have less friction with itself during inflation than a balloon having only a semi-crystalline polymer. It is believed that this is a reason for the improved compliance.

The wall 154 may have a thickness between about 55 microns and 70 microns, and may be about 65 microns as desired. Other thicknesses may be utilized as desired.

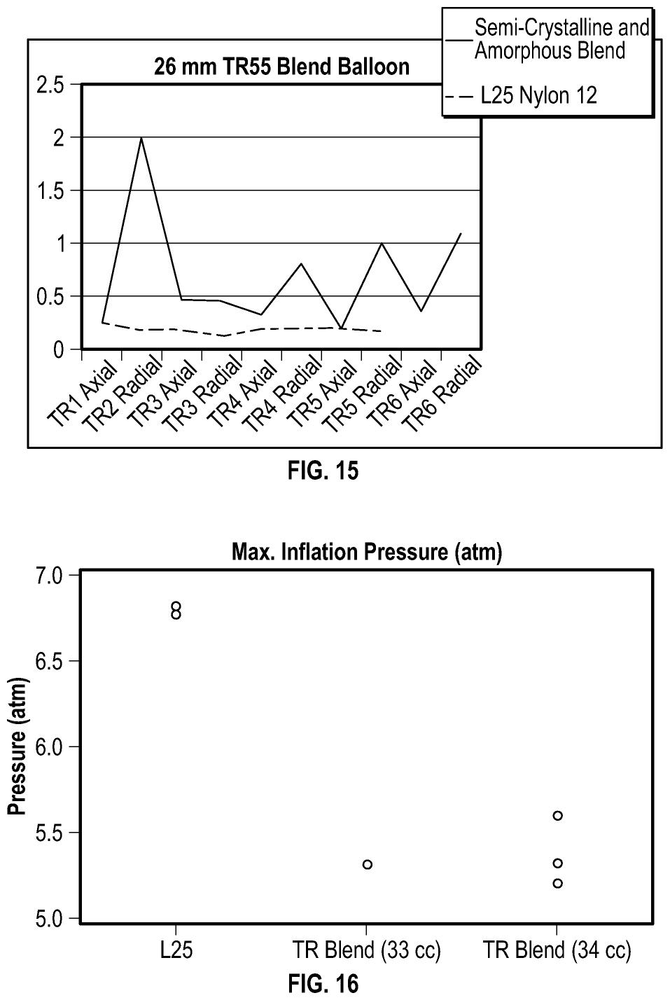

The balloon 150 having the blend of the semi-crystalline polymer and the amorphous polymer may further have increased tear strength in the radial dimension than in the axial dimension. FIG. 15, for example, illustrates a chart showing a metric of tear strength along the Y-axis relative to multiple trials of axial and radial tear strength on the X-axis. The balloons tested comprise a 26 millimeter diameter balloon. The blend comprises a semi-crystalline polymer that is about 75 percent by weight of the total weight of the blend, and the amorphous polymer that is about 25 percent by weight of the total weight of the blend. The balloon 150 having the blend is marked in solid lines and a balloon having only a semi-crystalline material (Grilamid L25) is shown in dashed lines. Significantly greater tear resistance in the radial dimension is shown. As such, if the balloon 150 having the blend were to accidentally burst then such a tear would be in the axial dimension, which may be desirable.

The balloon 150 having the blend of the semi-crystalline polymer and the amorphous polymer may further have a reduced maximum inflation pressure to deploy the implant than a balloon having only a semi-crystalline material (Grilamid L25). FIG. 16, for example, illustrates a chart showing maximum inflation pressure in atmospheres along the Y-axis for a balloon having only a semi-crystalline polymer (Grilamid L25) and two balloons 150 having the blend and inflated with 33 cubic centimeters and 34 cubic centimeters respectively. The blend comprises a semi-crystalline polymer that is about 75 percent by weight of the total weight of the blend, and the amorphous polymer that is about 25 percent by weight of the total weight of the blend.

The balloons 150 having the blend are shown to have a lower maximum inflation pressure by about 1.5 atmospheres. The maximum inflation pressures may be between about 5.2 and 5.8 atmospheres. Other amounts may be utilized in other examples.

Further properties of a balloon 150 having a blend comprising a semi-crystalline polymer that is about 75 percent by weight of the total weight of the blend, and the amorphous polymer that is about 25 percent by weight of the total weight of the blend, may include a burst pressure of about 10 atmospheres.

Properties of a balloon 150 having a blend comprising a semi-crystalline polymer that is about 90 percent by weight of the total weight of the blend, and the amorphous polymer that is about 10 percent by weight of the total weight of the blend, may include a burst pressure of about 11 atmospheres.

In examples, a burst pressure of the balloon may be at least about 7.5 atmospheres. The wall 154 may have a hoop strength of at least 22,000 pounds per square inch in examples. Other amounts may be utilized in examples.

An average compliance of a balloon 150 having a blend comprising a semi-crystalline polymer that is about 90 percent by weight of the total weight of the blend, and the amorphous polymer that is about 10 percent by weight of the total weight of the blend, may be about 0.5 millimeters per atmosphere.

A balloon having a blend of a semi-crystalline polymer and an amorphous polymer as disclosed herein may have a variety of benefits over a balloon including only a semi-crystalline polymer, including increased compliance, a linear compliance, lesser inflation pressure, reduced surface friction, and a greater radial tear strength than axial tear strength. Various other benefits may be provided.

A balloon having a blend of a semi-crystalline polymer and an amorphous polymer as disclosed herein may be utilized with any example of balloon or system disclosed herein. A balloon having a blend of a semi-crystalline polymer and an amorphous polymer as disclosed herein may be utilized with a compliant prosthetic valve in examples.

In examples, the balloon 150 may include at least two layers where the outer layer is comprised of the blend disclosed herein, and inner layer is comprised of a semi-crystalline polymer such as nylon 12 or another semi-crystalline polymer as disclosed herein. The outer layer may be a first layer and the inner layer may be a second layer positioned radially inward of the first layer. The blend may comprise the outer surface of the balloon and thus the outer surface may maintain a reduced friction and low tackiness surface. The outer blend layer may comprise 10 percent to 50 percent of total balloon wall thickness in examples, although other percentages may be utilized as desired.

The balloon may be utilized with a delivery catheter as disclosed herein, and may be coupled to a distal end portion of an elongate shaft of the delivery catheter. The balloon may be utilized in a similar manner as described with regard to FIGS. 11 and 12, and may be utilized to dilate a surface within a patient's body, which may comprise an expandable implant as desired. The balloon may be configured to dilate an expandable implant positioned on the wall of the balloon. The balloon may be utilized with a crimping device as disclosed herein.

The features of the examples disclosed herein may be implemented independently or in combination with other features disclosed herein. The various apparatuses of the system may be implemented independently.

As discussed, various forms of implants may be utilized with the examples disclosed herein, including prosthetic heart valves, or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The delivery apparatuses and the systems disclosed herein may be used in transcatheter aortic valve implantation (TAVI) or replacement of other native heart valves (e.g., mitral, tricuspid, or pulmonary). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

Features of examples may be modified, substituted, excluded, or combined across examples as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Example 1: A device for insertion within a portion of a patient's body, the device comprising: a balloon having a wall including an inner surface and a first layer and a second layer positioned adjacent to the first layer and not being thermally bondable to the first layer, wherein the balloon is expandable through pressure applied to the inner surface of the wall.

Example 2: The device of any example herein, in particular Example 1, wherein the first layer comprises a polyamide or co-polyamide.

Example 3: The device of any example herein, in particular Example 1 or Example 2, wherein the second layer comprises a polyester.

Example 4: The device of any example herein, in particular Examples 1-3, wherein the second layer comprises a polyethylene terephthalate, a polybutylene terephthalate, or a thermoplastic elastomer copolyester, or combinations thereof.

Example 5: The device of any example herein, in particular Examples 1-4, wherein the first layer is positioned radially inward of the second layer.

Example 6: The device of any example herein, in particular Example 5, further comprising a third layer positioned radially outward of the second layer and made of a material that is thermally bondable with the first layer.

Example 7: The device of any example herein, in particular Example 6, wherein the second layer or the third layer forms an outer surface of the wall.

Example 8: The device of any example herein, in particular Examples 1-7, wherein the second layer has less compliance than the first layer.

Example 9: The device of any example herein, in particular Examples 1-8, wherein the second layer comprises 25 percent to 50 percent of a thickness of the wall.

Example 10: The device of any example herein, in particular Examples 1-9, wherein the wall has an outer diameter compliance of between 10 percent growth per atmosphere and 16 percent growth per atmosphere between 4 atmospheres to 8 atmospheres when normalized by a diameter of the balloon.

Example 11: The device of any example herein, in particular Examples 1-10, wherein the wall has an outer diameter compliance of between 0.30 millimeters per atmosphere and 0.42 millimeters per atmosphere between 4 atmospheres to 8 atmospheres.

Example 12: The device of any example herein, in particular Examples 1-11, wherein a burst pressure of the balloon is at least about 7.5 atmospheres.

Example 13: The device of any example herein, in particular Examples 1-12, wherein the wall has a hoop strength of at least 24,000 pounds per square inch.

Example 14: The device of any example herein, in particular Examples 1-13, wherein an outer surface of the wall has a tapered profile.

Example 15: The device of any example herein, in particular Examples 1-14, wherein the balloon has an axial dimension and a radial dimension, and the wall has a weaker tear strength in the axial dimension than in the radial dimension.

Example 16: The device of any example herein, in particular Example 15, wherein the tear strength of the wall in the radial dimension is at least 20 percent higher than the tear strength in the axial dimension.

Example 17: The device of any example herein, in particular Examples 1-16, wherein the wall surrounds an interior chamber configured to receive fluid for expanding the balloon.

Example 18: The device of any example herein, in particular Examples 1-17, wherein the balloon is configured to dilate a surface within the patient's body.

Example 19: The device of any example herein, in particular Examples 1-18, wherein the balloon is configured to dilate an expandable implant positioned on the wall.

Example 20: The device of any example herein, in particular Examples 1-19, wherein the balloon is configured to couple to a delivery catheter for insertion within the patient's body.

Example 21: A system for deploying an implant to a portion of a patient's body, the system comprising: a delivery catheter including an elongate shaft; and a balloon coupled to the elongate shaft and configured for the implant to be positioned upon, and having a wall including an inner surface and a first layer and a second layer positioned adjacent to the first layer and not being thermally bondable to the first layer, wherein the balloon is expandable through pressure applied to the inner surface of the wall.

Example 22: The system of any example herein, in particular Example 21, wherein an outer surface of the wall has a tapered profile.

Example 23: The system of any example herein, in particular Example 21 or Example 22, wherein the first layer is thermally bondable to the elongate shaft.

Example 24: The system of any example herein, in particular Examples 21-23, wherein the first layer comprises a polyamide or co-polyamide.

Example 25: The system of any example herein, in particular Examples 21-24, wherein the second layer comprises a polyester.

Example 26: The system of any example herein, in particular Examples 21-25, wherein the second layer comprises a polyethylene terephthalate, a polybutylene terephthalate, or a thermoplastic elastomer copolyester, or combinations thereof.

Example 27: The system of any example herein, in particular Example 26, further comprising a third layer positioned radially outward of the second layer and made of a material that is thermally bondable with the first layer.

Example 28: The system of any example herein, in particular Example 27, wherein the second layer or the third layer forms an outer surface of the wall.

Example 29: The system of any example herein, in particular Examples 21-28, wherein the balloon has an axial dimension and a radial dimension, and the wall has a weaker tear strength in the axial dimension than in the radial dimension.

Example 30: The system of any example herein, in particular Examples 21-29, wherein the wall has an outer diameter compliance of between 0.30 millimeters per atmosphere and 0.42 millimeters per atmosphere between 4 atmospheres to 8 atmospheres.

Example 31: The system of any example herein, in particular Examples 21-30, wherein a burst pressure of the balloon is at least about 7.5 atmospheres.

Example 32: The system of any example herein, in particular Examples 21-31, wherein the wall has a hoop strength of at least 24,000 pounds per square inch.

Example 33: The system of any example herein, in particular Examples 21-32, further comprising the implant.

Example 34: The system of any example herein, in particular Example 33, wherein the implant comprises a prosthetic heart valve.

Example 35: The system of any example herein, in particular Example 33 or Example 34, wherein the implant has a linear outer diameter upon being crimped to the balloon.

Example 36: A method of delivering an implant to a portion of a patient's body, the method comprising: inserting a balloon into the patient's body, the balloon having a wall including an inner surface and a first layer and a second layer positioned adjacent to the first layer and not being thermally bondable to the first layer; and expanding the balloon through pressure applied to the inner surface of the wall to expand the implant at the portion of the patient's body.

Example 37: The method of any example herein, in particular Example 36, wherein the first layer comprises a polyamide or co-polyamide.

Example 38: The method of any example herein, in particular Example 36 or Example 37, wherein the second layer comprises a polyester.

Example 39: The method of any example herein, in particular Examples 36-38, wherein the second layer comprises a polyethylene terephthalate, a polybutylene terephthalate, or a thermoplastic elastomer copolyester, or combinations thereof.

Example 40: The method of any example herein, in particular Examples 36-39, wherein the first layer is positioned radially inward of the second layer.

Example 41: The method of any example herein, in particular Example 40, further comprising a third layer positioned radially outward of the second layer and made of a material that is thermally bondable with the first layer.

Example 42: The method of any example herein, in particular Example 41, wherein the second layer or the third layer forms an outer surface of the wall.

Example 43: The method of any example herein, in particular Examples 36-42, wherein the second layer has less compliance than the first layer.

Example 44: The method of any example herein, in particular Examples 36-43, wherein an outer surface of the wall has a tapered profile.

Example 45: The method of any example herein, in particular Examples 36-44, wherein the balloon has an axial dimension and a radial dimension, and the wall has a weaker tear strength in the axial dimension than in the radial dimension.

Example 46: A crimping device for an implant for implantation within a portion of a patient's body, the crimping device comprising: a compressive body having an inner surface surrounding a channel for the implant to be positioned within, the inner surface having a tapered profile and configured to be contracted to apply a compressive force to the implant within the channel to crimp the implant; and an actuator for contracting the inner surface.

Example 47: The crimping device of any example herein, in particular Example 46, wherein the channel is configured to receive a balloon therein, and the inner surface is configured to crimp the implant to the balloon.

Example 48: The crimping device of any example herein, in particular Example 46 or Example 47, wherein the tapered profile includes one or more linear surfaces.

Example 49: The crimping device of any example herein, in particular Examples 46-48, wherein the compressive body includes an opening for the implant to be passed through to be positioned within the channel.

Example 50: The crimping device of any example herein, in particular Examples 46-49, wherein a central portion of the inner surface includes the tapered profile.

Example 51: The crimping device of any example herein, in particular Examples 46-50, wherein the actuator comprises a lever.

Example 52: The crimping device of any example herein, in particular Example 51, wherein the lever is configured to be rotated to contract the inner surface.

Example 53: The crimping device of any example herein, in particular Examples 46-52, wherein the compressive body includes a rear face including a rear opening for the implant to be passed through to be positioned within the channel.

Example 54: The crimping device of any example herein, in particular Example 53, wherein the compressive body includes a front face facing opposite the rear face, the front face including a front opening for the implant to be passed through.

Example 55: The crimping device of any example herein, in particular Examples 46-54, further comprising a base that the compressive body is positioned upon.

Example 56: A method for crimping an implant for implantation within a portion of a patient's body, the method comprising: inserting an implant within a channel of a compressive body, the compressive body having an inner surface with a tapered profile surrounding the channel; and contracting the inner surface to apply a compressive force to the implant within the channel to crimp the implant.

Example 57: The method of any example herein, in particular Example 56, further comprising crimping the implant to a balloon.

Example 58: The method of any example herein, in particular Example 56 or Example 57, wherein the tapered profile includes one or more linear surfaces.

Example 59: The method of any example herein, in particular Examples 56-58, wherein the compressive body includes an opening for the implant to be passed through to be positioned within the channel.

Example 60: The method of any example herein, in particular Examples 56-59, wherein a central portion of the inner surface includes the tapered profile.

Example 61: The method of any example herein, in particular Examples 56-60, further comprising utilizing an actuator to contract the inner surface.

Example 62: The method of any example herein, in particular Example 61, wherein the actuator comprises a lever configured to be rotated to contract the inner surface.

Example 63: The method of any example herein, in particular Examples 56-62, wherein the compressive body includes a rear face including a rear opening for the implant to be passed through to be positioned within the channel.

Example 64: The method of any example herein, in particular Example 63, wherein the compressive body includes a front face facing opposite the rear face, the front face including a front opening for the implant to be passed through.

Example 65: The method of any example herein, in particular Examples 56-64, further comprising a base that the compressive body is positioned upon.

Example 66: A device for insertion within a portion of a patient's body, the device comprising: a balloon having a wall including a blend of a semi-crystalline polymer and an amorphous polymer and having an inner surface, wherein the balloon is expandable through pressure applied to the inner surface of the wall.

Example 67: The device of any example herein, in particular Example 66, wherein the semi-crystalline polymer comprises a semi-crystalline polyamide.

Example 68: The device of any example herein, in particular Example 66 or Example 67, wherein the semi-crystalline polymer comprises semi-crystalline nylon 12.

Example 69: The device of any example herein, in particular Examples 66-68, wherein the amorphous polymer comprises an amorphous polyamide.

Example 70: The device of any example herein, in particular Examples 66-69, wherein the amorphous polymer comprises a transparent amorphous nylon.

Example 71: The device of any example herein, in particular Examples 66-70, wherein the amorphous polymer comprises a transparent amorphous nylon with aromatic or cycloaliphatic units.

Example 72: The device of any example herein, in particular Examples 66-71, wherein the semi-crystalline polymer comprises at least 50 percent by weight of a total weight of the blend.

Example 73: The device of any example herein, in particular Examples 66-72, wherein the amorphous polymer comprises between 10 percent by weight and 40 percent by weight of a total weight of the blend.

Example 74: The device of any example herein, in particular Examples 66-73, wherein the semi-crystalline polymer has a melting temperature that is higher than a glass transition temperature of the amorphous polymer.

Example 75: The device of any example herein, in particular Examples 66-74, wherein a tensile modulus at room temperature of the amorphous polymer is greater than a tensile modulus at room temperature of the semi-crystalline polymer.

Example 76: The device of any example herein, in particular Examples 66-75, wherein the balloon has an axial dimension and a radial dimension, and the wall has a weaker tear strength in the axial dimension than in the radial dimension.

Example 77: The device of any example herein, in particular Examples 66-76, wherein the wall has a linear compliance upon the pressure being applied to the inner surface of the wall.

Example 78: The device of any example herein, in particular Examples 66-77, wherein the wall has an outer diameter compliance of between 17 percent growth per atmosphere and 21 percent growth per atmosphere between 4 atmospheres to 8 atmospheres when normalized by a diameter of the balloon.

Example 79: The device of any example herein, in particular Examples 66-78, wherein the wall has an outer diameter compliance of between 0.43 millimeters per atmosphere and 0.56 millimeters per atmosphere.

Example 80: The device of any example herein, in particular Examples 66-79, wherein a burst pressure of the balloon is at least about 7.5 atmospheres.

Example 81: The device of any example herein, in particular Examples 66-80, wherein the wall has a hoop strength of at least 22,000 pounds per square inch.

Example 82: The device of any example herein, in particular Examples 66-81, wherein the wall includes a first layer having the blend of the semi-crystalline polymer and the amorphous polymer, and includes a second layer positioned radially inward of the first layer, the second layer comprising nylon 12.

Example 83: The device of any example herein, in particular Examples 66-82, wherein the wall is blow molded.

Example 84: The device of any example herein, in particular Examples 66-83, wherein the balloon is configured to dilate an expandable implant positioned on the wall.

Example 85: The device of any example herein, in particular Examples 66-84, wherein the balloon is configured to couple to a delivery catheter for insertion within the patient's body.

Example 86: A system for deploying an implant to a portion of a patient's body, the system comprising: a delivery catheter including an elongate shaft; and a balloon coupled to the elongate shaft and configured for the implant to be positioned upon, and having a wall including a blend of a semi-crystalline polymer and an amorphous polymer and having an inner surface, wherein the balloon is expandable through pressure applied to the inner surface of the wall.

Example 87: The system of any example herein, in particular Example 86, wherein an outer surface of the wall has a tapered profile.

Example 88: The system of any example herein, in particular Example 86 or Example 87, wherein the semi-crystalline polymer comprises a semi-crystalline polyamide.

Example 89: The system of any example herein, in particular Examples 86-88, wherein the semi-crystalline polymer comprises semi-crystalline nylon 12.

Example 90: The system of any example herein, in particular Examples 86-89, wherein the amorphous polymer comprises a transparent amorphous nylon.

Example 91: The system of any example herein, in particular Examples 86-90, wherein the semi-crystalline polymer has a melting temperature that is higher than a glass transition temperature of the amorphous polymer.

Example 92: The system of any example herein, in particular Examples 86-91, wherein a tensile modulus at room temperature of the amorphous polymer is greater than a tensile modulus at room temperature of the semi-crystalline polymer.

Example 93: The system of any example herein, in particular Examples 86-92, wherein the balloon has an axial dimension and a radial dimension, and the wall has a weaker tear strength in the axial dimension than in the radial dimension.

Example 94: The system of any example herein, in particular Examples 86-93, further comprising the implant.

Example 95: The system of any example herein, in particular Example 94, wherein the implant comprises a prosthetic heart valve.

Example 96: A method of delivering an implant to a portion of a patient's body, the method comprising: inserting a balloon into the patient's body, the balloon having a wall including a blend of a semi-crystalline polymer and an amorphous polymer and having an inner surface; and expanding the balloon through pressure applied to the inner surface of the wall to expand the implant at the portion of the patient's body.

Example 97: The method of any example herein, in particular Example 96, wherein the balloon is positioned at a distal portion of an elongate shaft of a delivery catheter.

Example 98: The method of any example herein, in particular Example 96 or Example 97, wherein the semi-crystalline polymer comprises a semi-crystalline polyamide.

Example 99: The method of any example herein, in particular Examples 96-98, wherein the semi-crystalline polymer comprises semi-crystalline nylon 12.

Example 100: The method of any example herein, in particular Examples 96-99, wherein the amorphous polymer comprises a transparent amorphous nylon.

Example 101: The method of any example herein, in particular Examples 96-100, wherein the amorphous polymer comprises between 10 percent by weight and 40 by weight of a total weight of the blend.

Example 102: The method of any example herein, in particular Examples 96-101, wherein the semi-crystalline polymer has a melting temperature that is higher than a glass transition temperature of the amorphous polymer.

Example 103: The method of any example herein, in particular Examples 96-102, wherein a tensile modulus at room temperature of the amorphous polymer is greater than a tensile modulus at room temperature of the semi-crystalline polymer.

Example 104: The method of any example herein, in particular Examples 96-103, wherein the balloon has an axial dimension and a radial dimension, and the wall has a weaker tear strength in the axial dimension than in the radial dimension.

Example 105: The method of any example herein, in particular Examples 96-104, wherein the wall includes a first layer having the blend of the semi-crystalline polymer and the amorphous polymer, and includes a second layer positioned radially inward of the first layer, the second layer comprising nylon 12.

Any of the features of any of the examples, including but not limited to any of the first through 105 examples referred to above, is applicable to all other aspects and examples identified herein, including but not limited to any examples of any of the first through 105 examples referred to above. Moreover, any of the features of an example of the various examples, including but not limited to any examples of any of the first through 105 examples referred to above, is independently combinable, partly or wholly with other examples described herein in any way, e.g., one, two, or three or more examples may be combinable in whole or in part. Further, any of the features of the various examples, including but not limited to any examples of any of the first through 105 examples referred to above, may be made optional to other examples. Any example of a method can be performed by a system or apparatus of another example, and any aspect or example of a system or apparatus can be configured to perform a method of another aspect or example, including but not limited to any examples of any of the first through 105 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A system for deploying an implant to a portion of a patient's body, the system comprising:
a delivery catheter including an elongate shaft; and
a balloon coupled to the elongate shaft and configured for the implant to be positioned upon, and having a wall including an inner surface and a first layer and a second layer positioned adjacent to the first layer and not being thermally bondable to the first layer, the balloon having an axial dimension and a radial dimension,
wherein the balloon is expandable through pressure applied to the inner surface of the wall, and
wherein the wall has a weaker tear strength in the axial dimension than in the radial dimension.

2. The system of claim 1, wherein an outer surface of the wall has a tapered profile.

3. The system of claim 1, wherein the first layer is thermally bondable to the elongate shaft.

4. The system of claim 1, wherein the first layer comprises a polyamide or co-polyamide.

5. The system of claim 1, wherein the second layer comprises a polyester.

6. The system of claim 1, wherein the second layer comprises a polyethylene terephthalate, a polybutylene terephthalate, or a thermoplastic elastomer copolyester, or combinations thereof.

7. The system of claim 1, further comprising a third layer positioned radially outward of the second layer and made of a material that is thermally bondable with the first layer.

8. The system of claim 7, wherein the second layer or the third layer forms an outer surface of the wall.

9. The system of claim 1, wherein the second layer comprises 25 percent to 50 percent of a thickness of the wall.

10. The system of claim 1, wherein the wall has an outer diameter compliance of between 10 percent growth per atmosphere and 16 percent growth per atmosphere between 4 atmospheres to 8 atmospheres when normalized by a diameter of the balloon.

11. The system of claim 1, wherein the wall has an outer diameter compliance of between 0.30 millimeters per atmosphere and 0.42 millimeters per atmosphere between 4 atmospheres to 8 atmospheres.

12. The system of claim 1, wherein a burst pressure of the balloon is at least about 7.5 atmospheres.

13. The system of claim 1, wherein the wall has a hoop strength of at least 24,000 pounds per square inch.

14. The system of claim 1, wherein the tear strength of the wall in the radial dimension is at least 20 percent higher than the tear strength in the axial dimension.

15. The system of claim 1, further comprising the implant, wherein the implant comprises a prosthetic heart valve, and the balloon is configured to dilate the prosthetic heart valve when the prosthetic heart valve is positioned on the wall.

16. A system for deploying an implant to a portion of a patient's body, the system comprising:
a delivery catheter including an elongate shaft; and
a balloon coupled to the elongate shaft and configured for the implant to be positioned upon, and having a wall including a blend of a semi-crystalline polymer and an amorphous polymer and having an inner surface, the balloon having an axial dimension and a radial dimension,
wherein the balloon is expandable through pressure applied to the inner surface of the wall, and
wherein the wall has a weaker tear strength in the axial dimension than in the radial dimension.

17. The system of claim 16, wherein the semi-crystalline polymer comprises a semi-crystalline polyamide.

18. The system of claim 16, wherein the semi-crystalline polymer comprises semi-crystalline nylon 12.

19. The system of claim 16, wherein the amorphous polymer comprises a transparent amorphous nylon.

20. The system of claim 16, wherein the semi-crystalline polymer has a melting temperature that is higher than a glass transition temperature of the amorphous polymer.
